(19) United States
(12) Patent Application Publication
(10) Pub. No.: US 2022/0323719 A1
(43) Pub. Date: Oct. 13, 2022

(54) AXIAL AND ROTATIONAL LOCKING OF DUAL CATHETER SYSTEMS

(71) Applicant: Medtronic, Inc., Minneapolis, MN (US)

(72) Inventor: Jay T. Rassat, Otsego, MN (US)

(21) Appl. No.: 17/708,761

(22) Filed: Mar. 30, 2022

Related U.S. Application Data
(60) Provisional application No. 63/172,888, filed on Apr. 9, 2021.

(51) Int. Cl. A61M 25/00

(52) U.S. Cl. CPC A61M 25/0097; A61M 2025/0006; A61M 2205/0216; A61M 2207/00

(57) ABSTRACT

A device for axial and rotational locking of a dual catheter system and methods of the same. The system includes an outer catheter and an inner catheter adapted to be inserted into the outer catheter. The outer catheter extends along a catheter axis and includes an outer catheter shaft and an outer catheter hub. The inner catheter includes an inner catheter shaft and an inner catheter hub. The system also includes an intermediate hub located between the inner and outer catheters. The intermediate hub includes a deformable component positioned around at least a portion of the inner catheter. The deformable component is adapted to be inserted into the outer catheter hub such that the inner catheter is restricted, rotationally about and axially along the catheter axis, relative to the outer catheter.

This application claims the benefit of U.S. Provisional Patent Application Ser. No. 63/172,888 filed on Apr. 9, 2021, which is incorporated by reference herein in its entirety.

### FIELD

The present technology is generally related to dual catheter systems. For example, an inside catheter of the dual catheter may be manipulated within or in reference to an outside catheter (or sheath, further referred to herein as a catheter) of the dual catheter and, thereafter, the inside catheter may be locked relative to the outside catheter.

### BACKGROUND

Dual catheter systems (e.g., catheter-in-catheter, sheath-in-sheath, catheter-in-sheath) often include an inside catheter positioned within (e.g., received by) an outside catheter. The inside catheter may be rotated relative to the outside catheter (e.g., while the outside catheter maintains a fixed orientation) by the user. For example, during procedures the dual catheter system may be used to rotationally orient the inside catheter (e.g., relative to the outside catheter) into a desired position. After positioning the inside catheter relative to the outside catheter (e.g., into the desired position), the catheters often need to be held in position relative to one another, often with two hands.

By requiring two hands to maintain the position of the catheters relative to one another, the user may be limited. In other words, the user may not be able to perform other tasks in addition to manipulating the dual catheter. As such, it may be beneficial to have a dual catheter system that can lock the orientation of the inside catheter relative to the outside catheter such that the user may not need to use two hands.

### SUMMARY

The techniques of this disclosure generally relate to locking the rotational orientation and the axial position of an inner catheter relative to an outer catheter/sheath within which the inner catheter is positioned. For example, the inner catheter (or components proximate thereto) may include features that interact with the outer catheter/sheath to restrict movement of the inner catheter relative to the outer catheter. In other words, the inner catheter may be configured to be temporarily fixed relative to the outer catheter. Once the inner catheter is fixed relative to the outer catheter, the two catheters may be used in combination, e.g., with a single hand.

In one aspect, the present disclosure provides an apparatus including an outer catheter, an inner catheter, and an intermediate hub located between the inner and outer catheters. The outer catheter may include an outer catheter shaft and an outer catheter hub. The outer catheter may define an outer catheter passageway extending along a catheter axis. The outer catheter hub may be located at a proximal end of the outer catheter shaft. The inner catheter may include an inner catheter shaft and an inner catheter hub located at a proximal end of the inner catheter shaft. The inner catheter shaft may be adapted to be inserted into the outer catheter. The intermediate hub may include a deformable component positioned around at least a portion of the inner catheter shaft. The deformable component may be adapted to be inserted into the outer catheter hub such that the inner catheter is restricted, rotationally about and axially along the longitudinal axis, relative to the outer catheter.

In one or more embodiments, the outer catheter passageway at the outer catheter hub may define a constant diameter.

In one or more embodiments, the deformable component may extend from an outer surface of the inner catheter shaft by a thickness of less than or equal to 10 mm.

In one or more embodiments, the deformable component may extend along a length of the inner catheter shaft for a distance of about 15-20 mm.

In one or more embodiments, the deformable component may extend between a proximal region and a distal region. The deformable component may taper inwardly from the proximal region to the distal region.

In one or more embodiments, the deformable component may completely surround the inner catheter shaft.

In one or more embodiments, the deformable component may be inserted into the outer catheter hub by a distance of about 5-10 mm.

In one or more embodiments, the inner catheter may define an inner catheter passageway.

In one or more embodiments, the deformable component may include an elastomeric material.

In another aspect, the disclosure provides an intermediate hub including a body portion and a deformable component. The body portion may extend between a proximal end and a distal end. The body portion may be adapted to be manipulated by a user. The deformable component may be located at the distal end of the body portion and may be positioned around at least a portion of an inner catheter shaft. The deformable component may be adapted to be inserted into an outer catheter such that the inner catheter shaft is restricted, rotationally about and axially along a longitudinal axis, relative to the outer catheter.

In one or more embodiments, the deformable component may completely surround the inner catheter shaft.

In one or more embodiments, the deformable component may extend from an outer surface of the inner catheter shaft by a thickness of less than or equal to 10 mm.

In one or more embodiments, the deformable component may extend along a length of the inner catheter shaft for a distance of about 15-20 mm.

In one or more embodiments, the deformable component may extend between a proximal region and a distal region. The deformable component may taper inwardly from the proximal region to the distal region.

In one or more embodiments, the deformable component may be inserted into the outer catheter by a distance of about 5-10 mm.

In one or more embodiments, the deformable component may include an elastomeric material.

In one or more embodiments, the inner catheter shaft may define an inner catheter passageway.

In yet another aspect, the disclosure provides a method including providing an intermediate hub on an inner catheter. The inner catheter may include an inner catheter shaft and an inner catheter hub located at a proximal end of the inner catheter shaft. The intermediate hub may include a deformable component positioned around at least a portion of the inner catheter shaft. The method may also include inserting the inner catheter into an outer catheter passageway of an outer catheter. The catheter passageway may extend along a catheter axis. The outer catheter may include an outer catheter shaft and an outer catheter hub. The outer catheter hub may be located at a proximal end of the outer catheter shaft. Further, the method may include positioning the inner catheter rotationally about the catheter axis relative to the outer catheter and axially along the catheter axis relative to the outer catheter. The method may further include inserting the deformable component of the intermediate hub into the outer catheter to fix the axial and rotational position of the inner catheter relative to the outer catheter.

In one or more embodiments, providing the intermediate hub on the inner catheter may include positioning the intermediate hub over a distal end of the inner catheter shaft and sliding the intermediate hub to the inner catheter hub.

In one or more embodiments, the method may also include unlocking the intermediate hub from the inner catheter after inserting the inner catheter into the outer passageway of the outer catheter.

In one or more embodiments, the method may include moving the intermediate hub along the inner catheter shaft prior to inserting the deformable component into the outer catheter.

In one or more embodiments, the method may also include locking the intermediate hub to the outer catheter after fixing the inner and outer catheters relative to one another.

In one or more embodiments, inserting the deformable component into the outer catheter may include compressing the deformable component between the inner catheter shaft and the outer catheter.

In one or more embodiments, the method may also include delivering an implantable lead comprising one or more electrodes through the inner catheter.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic diagram that illustrates an inner catheter, an outer catheter, and an intermediate hub therebetween.

FIG. 2 is a cross-sectional view of the intermediate hub of FIG. 1 inserted within the outer catheter.

FIG. 3 is a schematic diagram that illustrates a dual catheter system showing an intermediate device attached to an inner device and relative to an outer device.

FIG. 4. is a schematic diagram that illustrates the dual catheter system of FIG. 3 showing the intermediate device positioned between the inner and outer devices.

FIG. 5 is a schematic diagram that illustrates the dual catheter system of FIG. 3 showing the intermediate device inserted into the outer device.

FIG. 6 is a flow diagram that illustrates one example of a method for positioning and locking an inner catheter relative to an outer catheter.

### DETAILED DESCRIPTION

The present disclosure describes an inner catheter that is positioned within an outer catheter and configurable such that the inner catheter may be fixed relative to the outer catheter using an intermediate hub. For example, the intermediate hub may include a deformable material (e.g., an elastomeric material, a polymer, rubber, a foam, etc.) surrounding at least a portion of the inner catheter shaft, which may be compressed between the inner and outer catheters to fix the relative positions thereof. Specifically, the rotational and axially position of the inner catheter may be fixed relative to the outer catheter.

The intermediate hub may be inserted over the inner catheter (e.g., over the distal end of the inner catheter shaft) such that the deformable material slides over the inner catheter shaft. In some embodiments, the intermediate hub may be removably coupled or lockable to the inner catheter hub. The inner catheter may be inserted into the outer catheter by inserting a distal end of the inner catheter through a proximal end of the outer catheter (e.g., within a passageway of the outer catheter). Further, the intermediate hub may be located between the inner and outer catheters (e.g., proximate the proximal end of the inner catheter) and adapted to be inserted into the outer catheter (e.g., the proximal end of the outer catheter).

When the deformable material is inserted into the outer catheter, the deformable material may be compressed between the inner catheter (e.g., an outer surface thereof) and the outer catheter (e.g., an inner surface thereof) such that rotational and axial movement of the inner catheter is fixed relative to the outer catheter. For example, the deformable material may compress between the inner and outer catheters to form a friction fit therebetween and restrict movement therebetween. The friction fit between the inner and outer catheters may restrict relative movement (e.g., both axial and rotational movement) between the inner and outer catheters. In other words, the inner catheter may be rotationally and axially (e.g., linearly) fixed relative to the outer catheter such that the inner catheter may only rotate or move axially if the outer catheter rotates or moves linearly. As such, the inner catheter may be locked into a desired orientation relative to the outer catheter, and both can be controlled, e.g., with a single hand. Fixing the orientation of the inner catheter relative to the outer catheter may be beneficial in procedures including lead delivery, therapy delivery, physiologic pacing (e.g., left bundle branch, His bundle branch, etc.), etc.

As used herein, the term “or” refers to an inclusive definition, for example, to mean “and/or” unless its context of usage clearly dictates otherwise. The term “and/or” refers to one or all of the listed elements or a combination of at least two of the listed elements.

As used herein, the phrases “at least one of” and “one or more of” followed by a list of elements refers to one or more of any of the elements listed or any combination of one or more of the elements listed.

As used herein, the terms “coupled” or “connected” refer to at least two elements being attached to each other either directly or indirectly. An indirect coupling may include one or more other elements between the at least two elements being attached. Either term may be modified by “operatively” and “operably,” which may be used interchangeably, to describe that the coupling or connection is configured to allow the components to interact to carry out described or otherwise known functionality.

As used herein, any term related to position or orientation, such as “proximal,” “distal,” “end,” “outer,” “inner,” and the like, refers to a relative position and does not limit the absolute orientation of an embodiment unless its context of usage clearly dictates otherwise.

All scientific and technical terms used herein have meanings commonly used in the art unless otherwise specified. The definitions provided herein are to facilitate understanding of certain terms used frequently herein and are not meant to limit the scope of the present disclosure.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

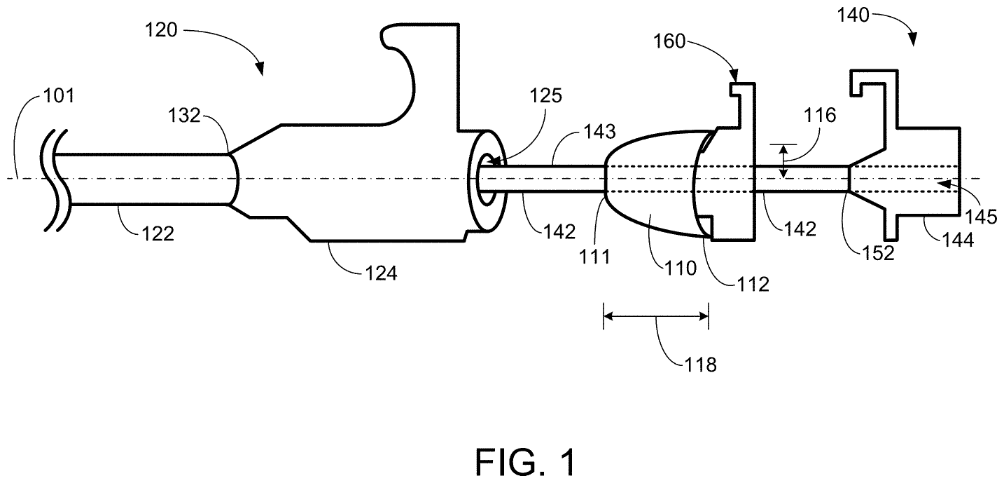

FIG. 1 illustrates an outer catheter 120 positioned relative to, but not connected to, an inner catheter 140. The outer catheter 120 may include an outer catheter shaft 122 and an outer catheter hub 124. The outer catheter hub 124 may be located at a proximal end 132 of the outer catheter shaft 122. The outer catheter hub 124 may serve as a rigid structure to more easily handle and guide elements into the outer catheter 120, while the outer catheter shaft 122 may define a more flexible structure. Further, the outer catheter 120 may define an outer catheter passageway 125 extending along a catheter axis 101 (e.g., through the outer catheter shaft 122 and the outer catheter hub 124). In other words, the outer catheter 120 may define an annular cross-sectional shape and be adapted to receive another device (e.g., the inner catheter 140). It is noted that the catheter axis 101 may not necessarily extend along a straight line because the catheter may be flexible and the catheter axis 101 extends along a centerpoint of the outer catheter passageway 125, e.g., through curves and bends of the outer catheter 120.

The outer catheter 120 may act as a sheath or cover for the inner catheter 140. For example, the inner catheter 140 may be adapted to be inserted into the outer catheter 120 (e.g., through the outer catheter passageway 125) such that the outer catheter 120 may serve as a guide for the inner catheter 140. Specifically, a distal end of the inner catheter 140 may be inserted into the proximal end of the outer catheter 120. Further, the inner catheter 140 may be inserted into the outer catheter 120 (e.g., until the proximal end 152 of the inner catheter 140 is closer to the proximal end 132 of the outer catheter 120). Additionally, the inner catheter 140 may be rotated relative to the outer catheter 120, while being inserted therein, to position the inner catheter 140 in a desired orientation rotationally and axially.

The inner catheter 140 may include an inner catheter shaft 142 and an inner catheter hub 144 located at a proximal end 152 of the inner catheter shaft 142. The inner catheter hub 144 may serve as a rigid structure to more easily handle, while the inner catheter shaft 142 may define a more flexible structure. Further, in one or more embodiments, the inner catheter 140 may define an inner catheter passageway 145 (e.g., through the inner catheter shaft 142 and the inner catheter hub 144). For example, the inner catheter passageway 145 may be utilized to deliver an implantable lead including one or more electrodes, to deliver therapy, to deliver physiologic pacing, to deliver other medical devices, to deliver pharmaceuticals, etc. In one or more embodiments, the inner catheter 140 may not include a lumen. For example, the outer catheter 120 may include a steerable outer sheath and the inner catheter 140 may include an inner ablation catheter (e.g., conventional ablation, cryoablation, etc.).

Additionally, an intermediate hub 160 may be positioned between the inner and outer catheters 140, 120. The intermediate hub 160 may include a deformable component 110 positioned around at least a portion of the inner catheter shaft 142 (e.g., on an outer surface of the inner catheter shaft 142). In one or more embodiments, the deformable component 110 may only surround a portion of the inner catheter shaft 142. In other embodiments, the deformable component 110 may completely surround the inner catheter shaft 142. The intermediate hub 160 may slide relative to (i.e., along) the inner catheter shaft 142 between the inner and outer catheters 140, 120 (e.g., except when the deformable component 110 is compressed into the outer catheter 120 as described herein).

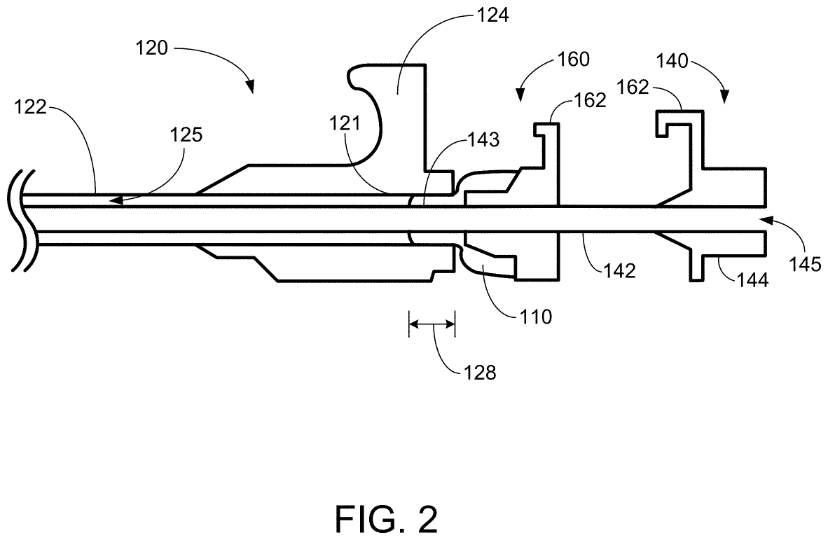

As shown in the cross-sectional view of FIG. 2, the deformable component 110 may be adapted to be inserted into the outer catheter 120 (e.g., the outer catheter hub 124). When the deformable component 110 is inserted into and compressed within the outer catheter 120, the inner catheter 140 may be restricted from moving relative to the outer catheter 120 (e.g., rotationally and axially). In other words, the inner catheter 140 may be restricted from rotating about the catheter axis 101 relative to the outer catheter 120 and may be restricted from moving axially along the catheter axis 101 relative to the outer catheter 120. For example, the deformable component 110 may compress between the outer surface 143 of the inner catheter shaft 142 and an inner surface 121 of the outer catheter 120 in such a way that the inner catheter 140 is restricted from moving relative to the outer catheter 120 (e.g., the inner catheter 140 may be squeezed such that it cannot move). Specifically, the deformable component 110 may form a friction fit between the inner and outer catheters 140, 120 to prevent movement therebetween. In one or more embodiments, the outer catheter 120 may include, e.g., a molded relief portion (e.g., underneath) that may allow the deformable component 110 to squeeze into the outer catheter 120 (e.g., the outer catheter hub 124). It is noted that while the deformable component 110 may prevent rotational and axial movement between the inner and outer catheters 140, 120, the intermediate hub 160 may still be removed from the outer catheter 120 (but, e.g., the deformable component 110 prevents the intermediate hub 160 from being inserted further into the outer catheter 120).

Therefore, the inner catheter 140 may be positioned rotationally (e.g., about the catheter axis 101) relative to the outer catheter 120 and axially (e.g., along the catheter axis 101) relative to the outer catheter 120 into a desired position. After the inner catheter 140 is in the desired rotational and axial position relative to the outer catheter 120, the deformable component 110 may freely move or slide along the inner catheter shaft 142 towards the outer catheter 120 to be inserted into the outer catheter 120. Once the deformable component 110 is inserted into the outer catheter 120, the inner catheter 140 is rotationally and axially locked or fixed into place (e.g., a locked configuration) relative to the outer catheter 120 (e.g., in the desired position).

The deformable component 110 may extend between a proximal region 112 and a distal region 111. Further, the deformable component 110 may extend along a length of the inner catheter shaft 142 for a distance 118 of about greater than or equal to 10 mm, greater than or equal to 15 mm, etc. and/or less than or equal 25 mm, less than or equal to 20 mm, etc. (e.g., measured between the proximal and distal regions 112, 111). When the intermediate hub 160 is inserted into the outer catheter 120 such that the inner and outer catheters 140, 120 are rotationally and axially fixed relative to one another, the deformable component 110 may be inserted into the outer catheter 120 (e.g., the outer catheter hub 124) by a distance 128 of about greater than or equal to 3 mm, greater than or equal to 5 mm, etc. and/or less than or equal to 15 mm, less than or equal to 10 mm, etc. (e.g., as shown in FIG. 2). The deformable component 110 may extend from the outer surface 143 of the inner catheter 140 by a thickness 116 of less than or equal to greater than or equal to 3 mm, greater than or equal to 5 mm, etc. and/or less than or equal to 25 mm, less than or equal to 20 mm, less than or equal to 15 mm, less than or equal to 10 mm, etc. It is noted that these dimensions may apply to 6 F to 1° F. sized catheters/sheath devices and for 16 F sized catheters/sheath devices, the above-described dimensions may be greater. For example, in one or more embodiments, the outer catheter 120 may define a 16 F ID and an inner catheter 140 may define a 12 F to 15 F size and, therefore, the length and thickness of the deformable component 110 may be greater.

The thickness 116 may be such that the deformable component 110 may be wider than an opening of the outer catheter 120 through which the inner catheter 140 is inserted and allows the deformable component 110 to be advanced into the outer catheter 10 while compressing the deformable component 110. In one or more embodiments, the deformable component 110 may taper inwardly from the proximal region 112 to the distal region 111. For example, the deformable component 110 may include a taper such that the deformable component 110 may be inserted into the outer catheter passageway 125 (e.g., to ease the deformable component 110 through an opening that may be smaller than an overall diameter of the deformable component 110). Specifically, the thickness of the deformable component at the distal region 111 may be smaller than the opening of the outer catheter 120 and the thickness of the deformable component at the proximal region 112 may be larger than the opening of the outer catheter 120. Further, the tapered shape of the deformable component 110 may assist in providing a seal between the inner and outer catheters 140, 120, by gradually compressing the deformable component 110 between the inner catheter 140 and the outer catheter 120. In some embodiments, the deformable component 110 may include a constant thickness throughout the length of the deformable component 110.

As shown in FIG. 2, the outer catheter 120 may define a constant diameter at the portion within which the deformable component 110 is inserted. Therefore, the deformable component 110 may be inserted into the fixed cross-sectional opening such that the deformable component 110 compresses, locking the inner catheter shaft 142 in position relative to the outer catheter shaft 122. Further, in one or more embodiments, any outer catheter 120 may be used in combination with the intermediate hub 160 to lock the position of the inner catheter 140 relative to the outer catheter 120. In other words, the intermediate hub 160 including the deformable component 110 may be a separate accessory from the outer catheter 120 and may be used with any outer catheter having an opening large enough to compress the deformable component 110.

The deformable component 110 may include any suitable material. For example, the deformable component 110 may include (e.g., be formed of) an elastomeric material, a polymer, a rubber, an open or closed cell foam, silicone, or similar material with deformable/conforming properties, etc. The deformable component 110 may be described as a material that is, e.g., soft, low modulus, compliant, compressible, auxetic or Poisson structured, etc. In one or more embodiments, the deformable component 110 may be described as compressible or not compressible. In other words, the deformable component 110 may include any material that allows for the deformable component 110 to be inserted into the outer catheter 120 and lock the rotational position between the inner and outer catheters 140, 120.

The deformable component 110 may be formed with the intermediate hub 160 in any suitable way. For example, in one or more embodiments, the deformable component 110 may be overmolded onto the body portion of the intermediate hub 160. In other embodiments, the deformable component 110 may be coupled with the intermediate hub 160 via adhesive. In further yet embodiments, the deformable component 110 may be coupled to the intermediate hub 160 by mechanical locking or fitment, chemical bonding, heat shrunk or reflowed, or through deposition mechanisms.

Also, in one or more embodiments, the intermediate hub 160 may be removably coupled or locked to the inner catheter hub 144 prior to positioning the inner catheter 140 relative to the outer catheter 120. The intermediate hub 160 may be removably coupled to the inner catheter hub 144 in any suitable way. For example, the inner catheter hub 144 and the intermediate hub 160 may include corresponding tabs 162 that removably couple them together. In one or more embodiments, the intermediate hub 160 may be unlocked or uncoupled from the inner catheter hub 144 so that the intermediate hub 160 can slide along the inner catheter shaft 142 before the deformable component 110 is compressed into the outer catheter 120. The inner catheter 140 may be positioned relative (e.g., rotationally and/or axially) to the outer catheter 120 before or after the intermediate hub 160 is unlocked or uncoupled from the inner catheter hub 144. Additionally, the intermediate hub 160 may be coupled to the outer catheter 120 after the deformable component 110 is inserted into the outer catheter 120.

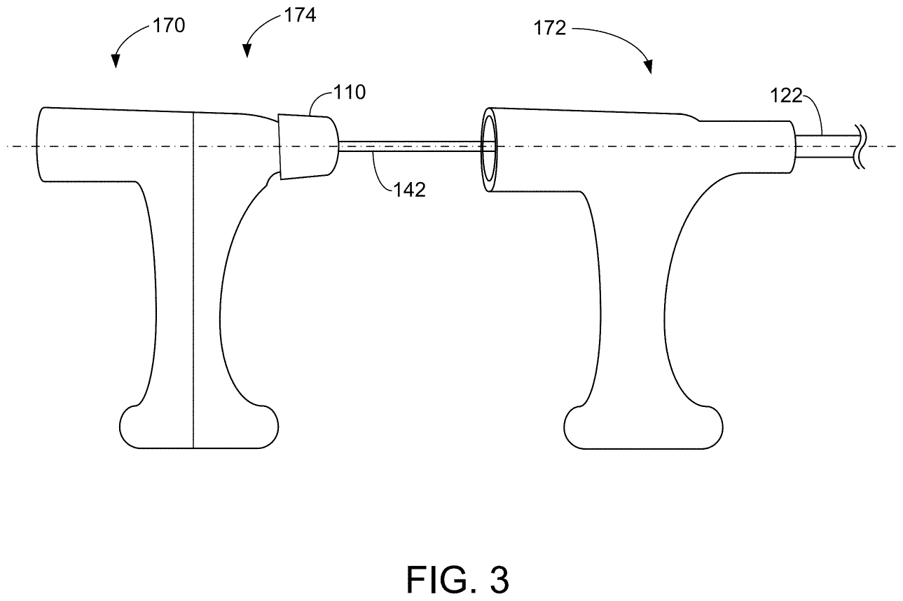

FIGS. 3-5 illustrate each of the inner and outer catheters 140, 120 included within a device. For example, an inner device 170 may include the inner catheter 140 and an outer device 172 may include the outer catheter 120. The devices may be more user friendly by providing features that are adapted to be held by the user. For example, the devices may include a body portion and a handle. Similarly, the intermediate hub 160 may be included within an intermediate device 174 that is positioned between the inner and outer devices 170, 172 (e.g., such that the deformable component 110 is around at least a portion of the inner catheter shaft 142). In other words, the devices may act as or replace the hubs described herein. In one or more embodiments, the intermediate device 174 may be a separate universal component that may be used with any existing suitable inner and outer catheter devices.

Each of the devices may include a body extending between a proximal end region and a distal end region. Also, each of the devices may include a handle extending from the body (e.g., to be easily utilized by the user). For example, the inner device 170 may be manipulated (e.g., rotated or moved axially) relative to the outer device 172 such that the inner catheter shaft 142 is moved relative to the outer catheter shaft 122 in the same way. Specifically, the inner device 170 may be moved to the desired rotational and axial position relative to the outer device 172 prior to the deformable component 110 being compressed within the outer device 172.

Furthermore, each of the devices may interact with one another to assist in operating the dual catheter system. For example, the inner device 170 may be lockably coupled to the intermediate device 174. Specifically, the inner device 170 and the intermediate device 174 may be initially provided together (e.g., the distal end region of the inner device 170 proximate the proximal end region of the intermediate device 174) and may be removably coupled (e.g., lockable) to one another (e.g., as shown in FIG. 3). The intermediate device 174 may be lockable with the inner device 170 in any suitable way. For example, the intermediate device 174 may be locked to the inner device 170 using a twist/positive locking feature (e.g., when the hub is turned, the intermediate device 174 may detach).

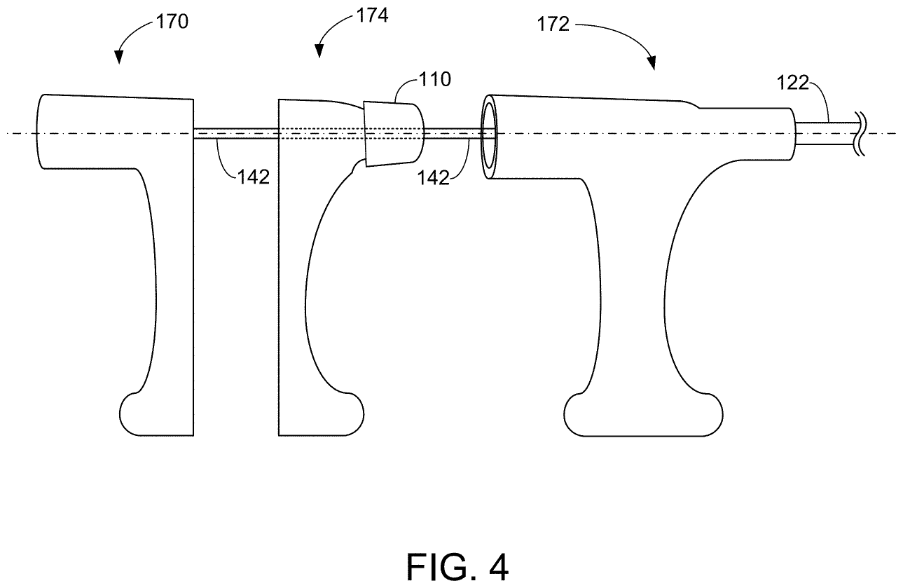

When the intermediate device 174 is unlocked and removed from the inner device 170, the intermediate device 174 may freely slide along the inner catheter shaft 142. For example, the intermediate device 174 is positioned between the inner and outer devices 170, 172 in FIG. 4, and the intermediate device 174 may move therebetween along the inner catheter shaft 142. The inner device 170 may also be moveable (e.g., rotationally and axially) relative to the outer device 172 to position the inner device 170 (and, thereby, the inner catheter) rotationally and axially relative to the outer device 172 (e.g., the outer catheter) into the desired position.

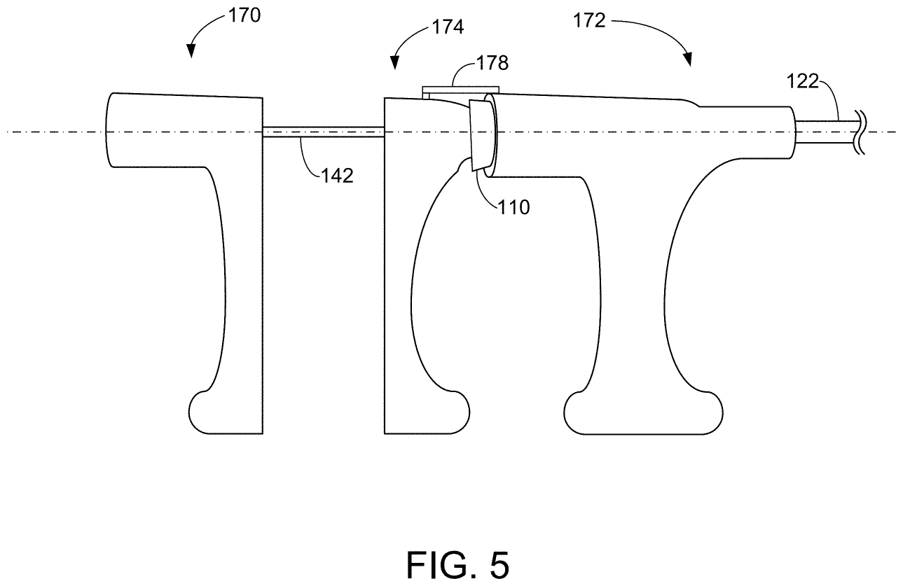

Additionally, once the intermediate device 174 is movable along the inner catheter shaft 142, the deformable component 110 may be inserted into the outer device 172 to fix the rotational and axial movement of the inner catheter relative to the outer catheter. After the deformable component 110 is compressed within the outer device 172 (e.g., within the outer catheter hub), in one or more embodiments, the intermediate device 174 may be removably coupled or locked to the outer device 172 (e.g., as shown in FIG. 5). For example, when the deformable component 110 is compressed within the outer catheter, the deformable component 110 may not be able to move further into the outer catheter (e.g., due to dimensional constraints), but the deformable component 110 may be removed from the outer catheter to release (e.g., no longer restrict) the relative rotational and axial movement between the inner and outer catheters. By locking the intermediate device 174 to the outer device 172 (e.g., after the deformable component 110 is compressed therein), the intermediate device 174 may not be inadvertently removed from (e.g., pulled out of) the outer device 172 and release the rotational and axial restrictions. The intermediate device 174 may be lockable with the outer device 172 in any suitable way. For example, a locking bracket 178 may extend over the deformable component 110 to create a positive lock or snap between the outer device 172 and the intermediate device 174.

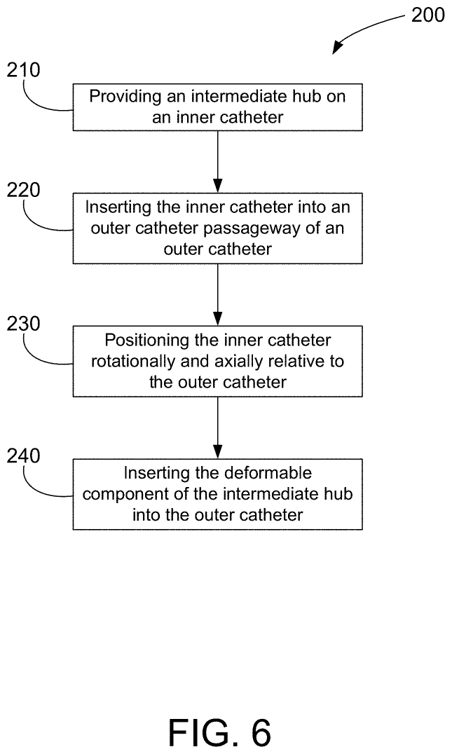

FIG. 6 shows one example of a method 200 for adjusting and locking a dual catheter system as described herein. For example, the method may include providing 210 an intermediate hub on an inner catheter (e.g., on an inner catheter shaft of the inner catheter) such that a deformable component of the intermediate hub may be positioned around at least a portion of the inner catheter shaft. Further, the method may include inserting 220 (e.g., compressing) the inner catheter into an outer catheter passageway of an outer catheter and positioning 230 the inner catheter relative to the outer catheter. For example, the inner catheter may be positioned rotationally (e.g., about a catheter axis) and axially (e.g., along the catheter axis) relative to the outer catheter. The method may also include inserting 240 the deformable component of the intermediate hub into the outer catheter to fix the axial and rotational position of the inner catheter relative to the outer catheter. In one or more embodiments, the method may further include delivering an implantable lead having one or more electrodes through the inner catheter (e.g., after the inner catheter is positioned relative to the outer catheter).

For example, in one or more embodiments, the dual catheter system (e.g., inner and outer catheters) with axial and rotational locking may be used for gaining venous access and entering the right atrium. Specifically, the outer catheter or sheath may be inserted from the insertion site (e.g., typically the subclavian vein) and traverse the venous system until the distal tip of the outer catheter (or sheath) enters the right atrium from the superior vena cava. The distal end shape of the outer catheter may likely allow for easy access to the right atrium (e.g., because it may define a sweeping large curve).

Further, the dual catheter system described herein may be used to navigate to the right ventricular septal wall fixation location. For example, the inner catheter may be inserted into the outer catheter/sheath and may be advanced until the tip of the inner catheter extends outside of the distal tip of the outer catheter. Together, the inner and outer catheters of the system may cross the tricuspid valve. The inner catheter may then extend to the desired depth of location, noting the orientation of the tip. If the tip of the inner catheter is pointing in the direction of the outflow track or away from the septum, the inner catheter may be rotated (or, e.g., the inner and outer catheters may be rotated together). Once the orientation of the two catheters (e.g., the inner catheter rotated and axial relative to the outer catheter) is at the desired location for reach, depth, and radial direction, the inner catheter orientation may be fixed with respect to the outer catheter by detaching and removing the fixation member (e.g., the intermediate hub including the deformable component) from the inner catheter hub. The fixation member (e.g., the deformable component) may next be advanced into the hub of the outer catheter/sheath to apply axial and radial force to fix the orientations of the inner and outer catheters with respect to one another. Keeping the location at the desired implant target, the lead may be advanced through the inner catheter and follow the fixation instructions per instructions for use (IFU) or training.

In one or more embodiments, providing the intermediate hub on the inner catheter may include positioning the intermediate hub over a distal end of the inner catheter shaft and sliding the intermediate hub to the inner catheter hub. In one or more embodiments, the method may also include unlocking the intermediate hub from the inner catheter. The intermediate hub may be unlocked from the inner catheter before or after the inner catheter is placed in the desired position relative to the outer catheter. The method may also include moving the intermediate hub along the inner catheter shaft prior to inserting the deformable component into the outer catheter.

In one or more embodiments, the method may also include locking the intermediate hub to the outer catheter after fixing the inner and outer catheters relative to one another. For example, compressing the deformable component into the outer catheter may stop the deformable component from be further inserted into the outer catheter, but it still may be possible to remove the deformable component by pulling it away from the outer catheter (i.e., this is how the deformable component may be removed to release the relative motion between the inner and outer catheters). Therefore, in one or more embodiments, the intermediate hub may be physically locked to the outer catheter to prevent inadvertent removal of the deformable component from the outer catheter.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

All references and publications cited herein are expressly incorporated herein by reference in their entirety for all purposes, except to the extent any aspect directly contradicts this disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

The singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

What is claimed is:

1. An apparatus comprising:
an outer catheter comprising an outer catheter shaft and an outer catheter hub, wherein the outer catheter defines an outer catheter passageway extending along a catheter axis, wherein the outer catheter hub is located at a proximal end of the outer catheter shaft;
an inner catheter comprising an inner catheter shaft and an inner catheter hub located at a proximal end of the inner catheter shaft, the inner catheter shaft is adapted to be inserted into the outer catheter; and
an intermediate hub located between the inner and outer catheters, wherein the intermediate hub comprises a deformable component positioned around at least a portion of the inner catheter shaft, wherein the deformable component is adapted to be inserted into the outer catheter hub such that the inner catheter is restricted, rotationally about and axially along the catheter axis, relative to the outer catheter.

2. The apparatus of claim 1, wherein the outer catheter passageway at the outer catheter hub defines a constant diameter.

3. The apparatus of claim 1, wherein the deformable component extends from an outer surface of the inner catheter shaft by a thickness of less than or equal to 10 mm.

4. The apparatus of claim 1, wherein the deformable component extends along a length of the inner catheter shaft for a distance of about 15-20 mm.

5. The apparatus of claim 1, wherein the deformable component extends between a proximal region and a distal region, wherein the deformable component tapers inwardly from the proximal region to the distal region.

6. The apparatus of claim 1, wherein the deformable component completely surrounds the inner catheter shaft.

7. The apparatus of claim 1, wherein the deformable component is inserted into the outer catheter hub by a distance of about 5-10 mm.

8. The apparatus of claim 1, wherein the deformable component comprises an elastomeric material.

9. An intermediate hub comprising:
a body portion extending between a proximal end and a distal end, wherein the body portion is adapted to be manipulated by a user; and
a deformable component located at the distal end of the body portion and positioned around at least a portion of an inner catheter shaft, wherein the deformable component is adapted to be inserted into an outer catheter such that the inner catheter shaft is restricted, rotationally about and axially along a catheter axis, relative to the outer catheter.

10. The intermediate hub of claim 9, wherein the deformable component completely surrounds the inner catheter shaft.

11. The intermediate hub of claim 9, wherein the deformable component extends from an outer surface of the inner catheter shaft by a thickness of less than or equal to 10 mm.

12. The intermediate hub of claim 9, wherein the deformable component extends along a length of the inner catheter shaft for a distance of about 15-20 mm.

13. The intermediate hub of claim 9, wherein the deformable component extends between a proximal region and a distal region, wherein the deformable component tapers inwardly from the proximal region to the distal region.

14. The intermediate hub of claim 9, wherein the deformable component is inserted into the outer catheter by a distance of about 5-10 mm.

15. The intermediate hub of claim 9, wherein the deformable component comprises an elastomeric material.

16. A method comprising:
providing an intermediate hub on an inner catheter, wherein the inner catheter comprises an inner catheter shaft and an inner catheter hub located at a proximal end of the inner catheter shaft, wherein the intermediate hub comprises a deformable component positioned around at least a portion of the inner catheter shaft;
inserting the inner catheter into an outer catheter passageway of an outer catheter, wherein the catheter passageway extends along a catheter axis, wherein the outer catheter comprises an outer catheter shaft and an outer catheter hub, wherein the outer catheter hub is located at a proximal end of the outer catheter shaft;
positioning the inner catheter rotationally about the catheter axis relative to the outer catheter and axially along the catheter axis relative to the outer catheter; and
inserting the deformable component of the intermediate hub into the outer catheter to fix the axial and rotational position of the inner catheter relative to the outer catheter.

17. The method of claim 16, wherein providing the intermediate hub on the inner catheter comprises positioning the intermediate hub over a distal end of the inner catheter shaft and sliding the intermediate hub to the inner catheter hub.

18. The method of claim 16, further comprising:
unlocking the intermediate hub from the inner catheter after inserting the inner catheter into the outer passageway of the outer catheter, and
moving the intermediate hub along the inner catheter shaft prior to inserting the deformable component into the outer catheter.

19. The method of claim 16, further comprising locking the intermediate hub to the outer catheter after fixing the inner and outer catheters relative to one another.

20. The method of claim 16, wherein inserting the deformable component into the outer catheter comprises compressing the deformable component between the inner catheter shaft and the outer catheter.
